(19) United States
(12) Patent Application Publication
(10) Pub. No.: US 2004/0180360 A1
(43) Pub. Date: Sep. 16, 2004

(54) Multivalent aptamer therapeutics with improved pharmacodynamic properties and methods of making and using the same

(76) Inventors: Charles Wilson, Concord, MA (US); David Epstein, Belmont, MA (US); Jeffrey Kurz, Arlington, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/718,833

(22) Filed: Nov. 21, 2003

Related U.S. Application Data
(60) Provisional application No. 60/428,102, filed on Nov. 21, 2002.
(60) Provisional application No. 60/469,628, filed on May 8, 2003.

(51) Int. Cl. C12Q 1/68; C07H 21/04

(52) U.S. Cl. 435/6; 536/23.5

(57) ABSTRACT

Materials and methods are provided for producing aptamer therapeutics having improved pharmacokinetic and pharmacodynamic properties, as well as increased target valency. The aptamers produced by the methods of the invention are useful as therapeutics for treating disease.

### REFERENCE TO RELATED APPLICATIONS

This application is related to and claims priority under 35 U.S.C. § 119(e) to U.S. Provisional Patent Application Serial No. 60/428,102, filed Nov. 21, 2002, and U.S. Provisional Patent Application Serial No. 60/469,628, filed May 8, 2003, each of which is herein incorporated by reference in its entirety.

### FIELD OF THE INVENTION

The invention relates generally to the field of nucleic acids and more particularly to aptamers having improved target valency, pharmacodynamic, and pharmacokinetic properties. The invention further relates to improving the therapeutic effectiveness of aptamer therapeutics.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block the targets' ability to function. Created by an in vitro selection process from pools of random sequence oligonucleotides (FIG. 1), aptamers have been generated for over 100 proteins, including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, and the like) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial (therapeutic) leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 kDa; antibody: 150 kDa), a weekly dose of aptamer may be delivered by injection in a volume of less than 0.5 ml. Aptamer bioavailability via subcutaneous administration is >80% in monkey studies (Tucker et al., J. Chromatography B. 732: 203-212, 1999). In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Given the advantages of aptamers as therapeutic agents, it would be beneficial to have materials and methods to improve the target valency, pharmacokinetic and pharmacodynamic properties of aptamer therapeutics. The present invention provides materials and methods to meet these and other needs.

### BRIEF DESCRIPTION OF THE DRAWINGS

FIG. 1 shows the in vitro aptamer selection (SELEX™) process from pools of random sequence oligonucleotides.

FIG. 2 illustrates various strategies for synthesis of high molecular weight PEG-nucleic acid conjugates.

FIG. 3 is a chromatographic trace of the synthesis of a 3′-5′-diPEGylated nucleic acid.

FIG. 4(A) illustrates PDGF bound to a bidentate aptamer ligand stabilized with an oligonucleotide splint;

FIG. 4(B) is a binding plot showing the proportion of bound bidentate aptamer to PDGF-BB with respective monomer controls.

FIG. 5 (A) is a binding plot showing the effect of 10 nM splint DNA on the affinity of a PDGF-BB bidentate aptamer;

FIG. 5(B) is a binding plot showing the effect of 100 nM splint DNA on the binding affinity of a PDGF-BB bidentate aptamer.

FIG. 6 illustrates the design of a TGFβ2 bidentate aptamer with various spacer compositions and lengths.

FIG. 7(A) is a binding plot showing the effect of various linker lengths and compositions on the binding of a TGFβ2 bidentate aptamer;

FIG. 7(B) is a plot of a competition assay of ³²P-labeled TGFβ2 aptamer with a TGFβ2 bidentate aptamer with various nucleotide linker lengths. The 5-20 nt linker showed a K_{comp}=0.3 nM and the 30-50 nt linkers showed a K_{comp}=3-4 nM, roughly a 10-fold decrease.

### SUMMARY OF THE INVENTION

The present invention provides high molecular weight PEG-derivatized nucleic acid (e.g., aptamer) conjugates with improved pharmacological and pharmacodynamic properties and methods for producing such conjugates.

In one embodiment, the present invention provides high molecular weight PEG-nucleic acid (e.g., aptamer) conjugates and methods for producing such conjugates using a homo-bifunctional PEG to form a high molecular weight dimer (i.e., a nucleic acid-PEG-nucleic acid conjugate).

In one embodiment, the present invention provides high molecular weight PEG-nucleic acid (e.g., aptamer) conjugates and methods for producing such conjugates using a bi-reactive nucleic acid (i.e., a nucleic acid bearing two reactive sites) with a mono-functional PEG to form a multiple PEGylated conjugate (i.e., a PEG-nucleic acid-PEG conjugate).

In one embodiment, the present invention provides oligonucleotide-splinted stabilized multivalent aptamers with enhanced ligand binding properties and methods for producing such conjugates.

In one embodiment, the present invention provides oligonucleotide-linked multivalent aptamers having improved ligand binding properties and methods for producing such conjugates.

In one embodiment, the materials and methods of the present invention can be used to generate aptamer molecule multimers that have specificity to a target.

In one embodiment, the aptamers of the present invention can be used as therapeutics in the prevention and/or treatment of diseases and disorders.

In one aspect, high molecular weight aptamer compositions of the invention include a nucleic acid having two or more aptamers, and a stabilizing moiety that is a linking moiety, wherein the linking moiety is not a nucleic acid molecule. In one embodiment, the linking moiety is polyalkylene glycol. Suitable polyalkylene glycols include, for example, polyethylene glycol (PEG). In some embodiments, the polyethylene glycol (PEG) linking moiety is multi-activated. For example, the PEG linking moiety is bi-activated. In one embodiment, high molecular weight aptamer compositions include a nucleic acid that has first and second aptamers. In this embodiment, the first and second aptamers are linked by a PEG linking moiety, such that the primary structure of the aptamer composition is a linear arrangement in which the first aptamer is linked to a first terminus of the PEG linking moiety and the second aptamer is linked to a second terminus of the PEG linking moiety. In some embodiments, the high molecular weight aptamer composition has a molecular weight selected from the group consisting of greater than 10 kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect of the invention, the high molecular weight aptamer compositions include a nucleic acid moiety having two or more aptamer domains joined by a linker domain, and a stabilizing moiety in which one or more polyalkylene glycol moieties attached to the linker domain. In one embodiment, the stabilization moiety is one or more polyalkylene glycol moieties attached to the linker domain. Suitable polyalkylene glycols include, for example, polyethylene glycol (PEG). In some embodiments, the high molecular weight aptamer composition has a molecular weight selected from the group consisting of greater than 10I kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect, the invention provides high molecular weight aptamer compositions that include a nucleic acid having two or more aptamer domains and a linker domain, and a stabilizing moiety that includes an oligonucleotide splint which hybridizes to at least a portion of the linker domain, wherein the oligonucleotide splint has a nucleotide sequence having at least 40 nucleotides. In one embodiment, the oligonucleotide splint hybridizes to at least 20 nucleotides of the linker domain. In some embodiments, the high molecular weight aptamer composition has a molecular weight selected from the group consisting of greater than 10 kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect of the invention, the high molecular weight aptamer compositions include a nucleic acid moiety having two or more aptamer domains and a linker domain, and a stabilizing moiety that includes an oligonucleotide splint that hybridizes to at least a portion of the linker domain, wherein the oligonucleotide splint has one or more polyalkylene glycol moieties attached thereto. Suitable polyalkylene glycols include, for example, polyethylene glycol (PEG). In one embodiment, the oligonucleotide splint hybridizes to at least 20 nucleotides of the linker domain. In one embodiment, the oligonucleotide splint has a nucleotide sequence having at least 40 nucleotides. In some embodiments, the high molecular weight aptamer composition has a molecular weight selected from the group consisting of greater than 10 kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect, the invention provides high molecular weight aptamer compositions that include a nucleic acid moiety having two or more aptamer domains and a linker domain, and a stabilizing moiety that includes an oligonucleotide splint which hybridizes to at least a portion of the linker domain, wherein at least one of the two or more aptamer domains is in the unbound state (i.e., not bound to a specific aptamer target). In one embodiment, the oligonucleotide splint hybridizes to at least 20 nucleotides of the linker domain. In one embodiment, the oligonucleotide splint has a nucleotide sequence having at least 40 nucleotides. In one embodiment, the oligonucleotide splint has one or more polyalkylene glycol moieties attached thereto. Suitable polyalkylene glycols include, for example, polyethylene glycol (PEG). In some embodiments, the high molecular weight aptamer composition has a molecular weight selected from the group consisting of greater than 10 kD, greater than 20 kD, greater than 40 kD and greater than 80 kD. Some high molecular weight aptamer compositions according to this aspect of the invention are capable of binding to platelet derived growth factor (PDGF). Some high molecular weight aptamer compositions according to this aspect of the invention are capable of binding to TGFβ2.

In another aspect, the invention provides high molecular weight aptamer compositions that include an aptamer, and two or more non-nucleic acid stabilizing moieties. Suitable stabilizing moieties include, for example, a polyalkylene glycol. In one embodiment, the stabilizing moiety is polyethylene glycol (PEG). In one embodiment, the aptamer is multi-activated. For example, the aptamer is bi-activated.

The present invention also provides therapeutic compositions. Therapeutic compositions according to the invention include the high molecular weight aptamer compositions described herein.

In another aspect, the present invention provides methods of improving the pharmacokinetic or pharmacodynamic properties of an aptamer therapeutic composition including the steps of introducing reactive groups in a nucleic acid aptamer, and reacting the reactive groups on the aptamer with reactive groups on a stabilizing moiety, thereby forming a stabilized high molecular weight therapeutic aptamer. In one embodiment, the reactive groups on the aptamer composition are amino groups at 5′ or 3′ ends of the aptamer introduced by modified phosphoramidite synthesis. In one embodiment, the stabilizing moiety is polyethylene glycol (PEG). In a further embodiment, the PEG is homo-bifunctional and the resulting aptamer is a dimer linked by a PEG linker. In one embodiment, the aptamer is multi-activated. For example, the aptamer is bi-activated. In one embodiment, the aptamer is bi-activated at the 5′ and 3′ termini. In one embodiment, the stabilizing moiety is a mono-activated PEG and the resulting aptamer is bi-PEGylated.

In another aspect, the present invention provides methods of treating disease in a subject comprising the steps of administering a therapeutically effective amount of a high molecular weight aptamer compositions described herein.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

PEG-Derivatized Nucleic Acids

Derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (PAG) moieties. Typical polymers used in the invention include poly(ethylene glycol) (PEG), also known as or poly(ethylene oxide) (PEO) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpoly(ethylene glycol), can also be represented as HO-PEG-OH, where it is understood that the -PEG- symbol represents the following structural unit: —CH₂CH₂O—(CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively nonreactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules. Polyalkylated compounds of the invention are typically between 5 and 80 kD in size. Other PAG compounds of the invention are between 10 and 80 kD in size. Still other PAG compounds of the invention are between 10 and 60 kD in size. For example, a PAG polymer may be at least 10, 20, 30, 40, 50, 60, or 80 kD in size. Such polymers can be linear or branched.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i.e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol. 2: 214-221, 2003).

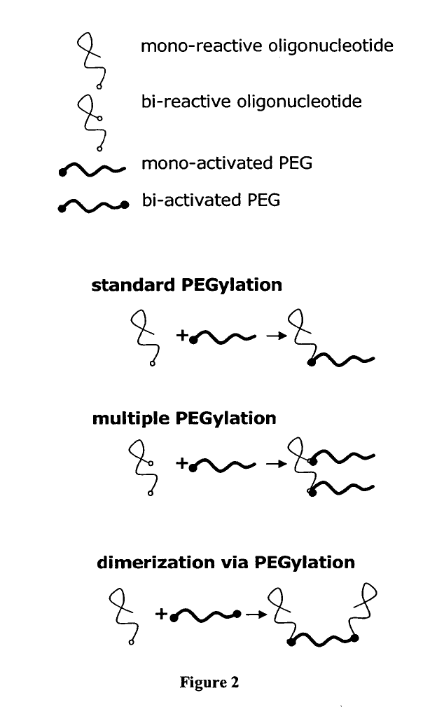

The present invention provides another cost effective route to the synthesis of high molecular weight PEG-nucleic acid (preferably, aptamer) conjugates including multiply PEGylated nucleic acids (as illustrated, e.g., in FIG. 2). The present invention also encompasses PEG-linked multimeric oligonucleotides, e.g., dimerized aptamers (as also illustrated, e.g., in FIG. 2).

High molecular weight compositions of the invention include those having a molecular weight of at least 10 kD. Compositions typically have a molecular weight between 10 and 80 kD in size. High molecular weight compositions of the invention are at least 10, 20, 30, 40, 50, 60, or 80 kD in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). Oligonucleotides can also be stabilizing moieties; such oligonucleotides can include modified nucleotides, and/or modified linkages, such as phosphothioates. A stabilizing moiety can be an integral part of an aptamer composition, i.e., it is covalently bonded to the aptamer. Alternatively, the stabilizing moiety can associate with the aptamer composition non-covalently, such as via hydrogen bonding or hybridization interactions between two oligonucleotides.

PEG-mediated Dimerization of Aptamers. Dimerization via a bi-functional PEG offers multiple potential benefits including (1) increased affinity in binding to dimeric targets, (2) increased avidity and decreased dissociation rate in binding to all targets, and (3) increased effective molecular weight with corresponding increased resistance to clearance via filtration.

Compositions of the invention include high molecular weight aptamer compositions in which two or more aptamers are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the aptamers together in one molecule, the polyalkylene glycol is said to be a linking moiety. In such compositions, the primary structure of the covalent molecule includes the linear arrangement aptamer-PAG-aptamer. One example is a composition having the primary structure aptamer-PEG-aptamer.

To produce the nucleic acid-PEG-nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mM and the reconstituted solution contains 200 mM NaHCO₃-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

PAG-derivatization of a reactive nucleic acid. High molecular weight PAG-nucleic acid-PAG conjugates can be prepared by reaction of a mono-functional activated PEG with a nucleic acid containing more than one reactive site. In a preferred embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 2. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully-, partially-, and un-conjugated species. FIG. 2 illustrates the two strategies for synthesizing PEGylated nucleic acid aptamers.

Multivalent Aptamers

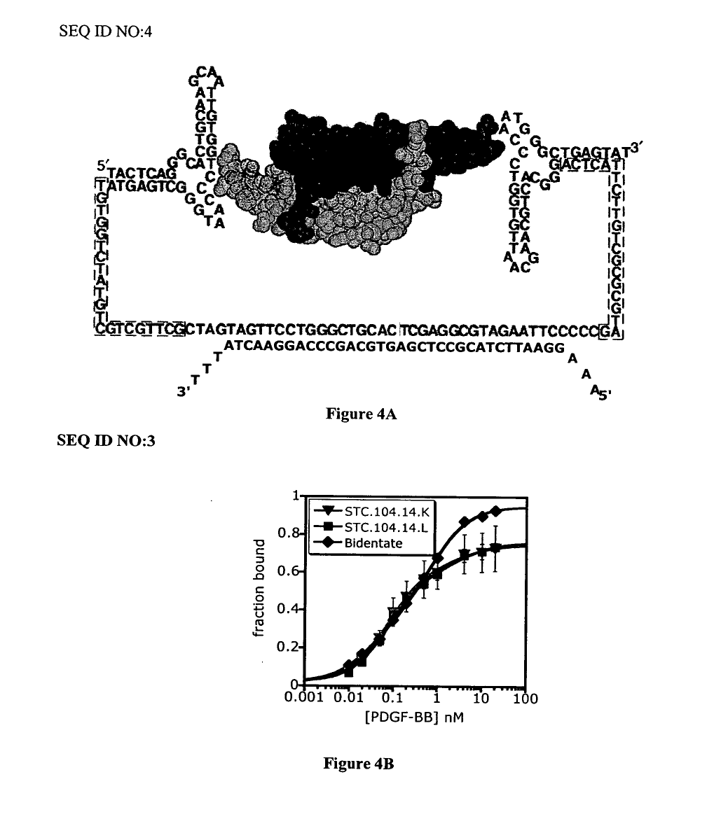

Also disclosed herein are splinted and non-splinted multivalent (e.g., bivalent or dimerized) aptamers. The splinted bivalent aptamers (illustrated, e.g., in FIG. 4A) are comprised of two oligonucleotides. The first oligonucleotide is comprised of two or more aptamer domains (e.g., target binding domains), which can be previously identified aptamers or regions of previously identified aptamers joined by a single-stranded linker domain. The second oligonucleotide (or “splint oligonucleotide”) is complementary and binds to a portion of the linker domain of the first oligonucleotide. The oligonucleotide splint can have a nucleic acid sequence that includes at least 40 nucleotides. When bound to the linking domain, the oligonucleotide splint preferably has at least twenty nucleotides hybridized to the linking domain. The oligonucleotide splint can also have one or more polyalkylene glycol moieties attached thereto. In some splinted, bivalent aptamer compositions, at least one of the two or more aptamer domains is in an unbound state (i.e., not bound to a specific target). Binding of the splint oligonucleotide, which is preferably DNA, to the first oligonucleotide is believed to increase stability by (1) providing some rigidity to the first oligonucleotide, (2) preventing the single-stranded region from interacting with the target binding regions, and/or (3) reducing rotational freedom.

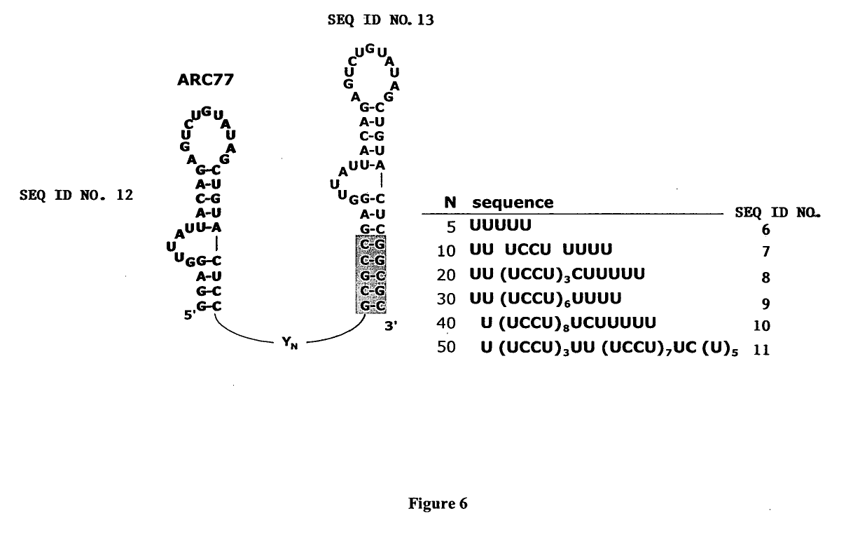

Non-splinted bivalent aptamers (illustrated, e.g., in FIG. 6) are comprised of a single oligonucleotide comprising two target binding domains (e.g., previously identified aptamers or regions of previously identified aptamers) joined by a single-stranded linker domain. The linking domain of these non-splinted bivalent aptamers can have one ore more polyalkylene glycol moieties attached thereto. Such PAGs can be of varying lengths and may be used in appropriate combinations to achieve the desired molecular weight of the composition.

Chelating ligands, typically bivalent or multivalent, are known to form more stable interactions with their binding partners than their corresponding monodentate, or monovalent, ligands. In the present invention, an oligonucleotide linker is used to link multiple aptamers to achieve a chelating effect with a bidentate or multivalent aptamer. The linker region can be used with or without a “splint oligonucleotide” to further stabilize the construct. The composition of the oligonucleotide linker can be of a heterogeneous sequence or it can be a poly U/C or poly A/G linker of various sequences and/or lengths. The effect of the various linker sequences and composition has desirable effects on aptamer ligand properties as described in Example 2 and Example 3.

Multivalent aptamers have improved pharmacokinetic and/or pharmacodynamic properties relative to monomeric aptamers. Both enthalpic and entropic effects contribute to the enhanced affinity of chelator-like multivalent aptamers. The enthalpic gain results from the several additional interactions formed between the bivalent ligand and its target. The entropic gain, in part, reflects the reduced entropic penalty associated with the formation of a 1:1 complex between bivalent ligand and target as compared to the binding of two monovalent ligands to the same target. Additionally, the “effective concentration” of the ligand is increased. For example, when a monomeric ligand dissociates from its target, it is released into bulk solution; however, when one of the liganding moieties of a chelate dissociates, its movement is constrained to within the proximity of the target by tethering to the bound cognate moiety.

The effect of a particular linker sequence can be influenced by both its chemical composition and length. A linker that is too short will clearly preclude the formation of a chelate. However, a linker that forms unfavorable steric and/or ionic interactions with the target will also negate the stabilizing effects of chelation. On the other hand, lengthening of the linker, beyond that necessary to span the distance between binding sites may reduce binding stability by diminishing the effective concentration of the ligand. Thus, it is often necessary to optimize linker compositions and lengths in order to maximize the affinity of a chelating ligand. Nevertheless, preliminary measurements with bidentate aptamers specific to two cytokine homodimers (PDGF and TGFβ2), as described in Example 2 and Example 3, indicate that linking aptamers has desirable effects on aptamer binding properties. The derivatized aptamers having improved pharmacokinetic and pharmacodynamic properties of the present invention can be initially obtained by the “Systematic Evolution of Ligands by EXponential Enrichment” (“SELEX™”) method as described below.

The SELEX™ Method

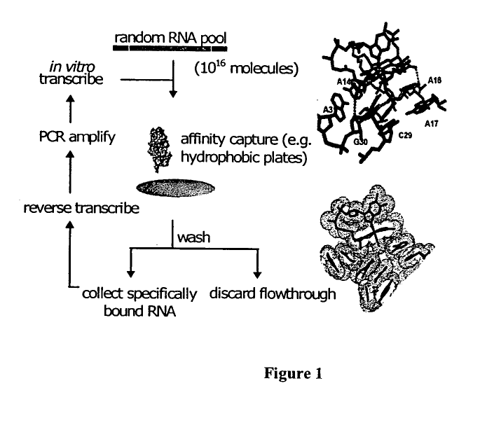

A suitable method for generating an aptamer is with the process entitled “Systematic Evolution of Ligands by EXponential Enrichment” (“SELEX™”) generally depicted in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores (described further below), sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs. See, e.g., U.S. Pat. Nos. 5,958,691; 5,660,985; 5,958,691; 5,698,687; 5,817,635; and 5,672,695, PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 □mole) scale synthesis will yield 1015-1016 individual template molecules, sufficient for most SELEX™ experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX™”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, see U.S. Pat. No. 5,580,737 which discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described in U.S. Pat. No. 5,660,985, which describes oligonucleotides containing nucleotide derivatives chemically modified at the 2′ of ribose, 5 position of pyrimidines and 8 positions of purines. U.S. Pat. No. 5,756,703 describes oligonucleotides containing various 2′-modified pyrimidines. U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanosine and the like. Modifications can also include 3′ and 5′ modifications such as capping. In preferred embodiments of the instant invention, the nucleic acid ligands are RNA molecules that are 2′-fluoro (2′-F) modified on the sugar moiety of pyrimidine residues.

The modifications can be pre- or post-SELEX™ process modifications. Pre-SELEX™ process modifications yield nucleic acid ligands with both specificity for their SELEX™ target and improved in vivo stability. Post-SELEX™ process modifications made to 2′-OH nucleic acid ligands can result in improved in vivo stability without adversely affecting the binding capacity of the nucleic acid ligand.

Other modifications are known to one of ordinary skill in the art. Such modifications may be made post-SELEX™ process (modification of previously identified unmodified ligands) or by incorporation into the SELEX™ process.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications allow the combination of a broad array of shapes and other properties, and the efficient amplification and replication of oligonucleotides with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). The use of 2′-fluoro-ribonucleotide oligomer molecules can increase the sensitivity of a nucleic acid sensor molecule for a target molecule by ten-to-one hundred-fold over those generated using unsubstituted ribo- or deoxyribooligonucleotides (Pagratis, et al., Nat. Biotechnol. 15:68-73 (1997)), providing additional binding interactions with a target molecule and increasing the stability of the secondary structure(s) of the nucleic acid sensor molecule (Kraus, et al., Journal of Immunology 160:5209-5212 (1998); Pieken, et al., Science 253:314-317 (1991); Lin, et al., Nucl. Acids Res. 22:5529-5234 (1994); Jellinek, et al. Biochemistry 34:11363-11372 (1995); Pagratis, et al., Nat. Biotechnol. 15:68-73 (1997)).

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase or modified T7 RNA polymerases and purified. In one example, the 5′-fixed:random:3′-fixed sequence is separated by random sequence having 30 to 50 nucleotides.

Pharmaceutical Compositions

The invention also includes pharmaceutical compositions containing the aptamer molecules of the present invention. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers of aptamer domains specifically bind.

For example, the target is a protein involved with a pathology, for example, the target protein causes the pathology.

Compositions of the invention can be used in a method for treating a patient having a pathology. The method involves administering to the patient a composition comprising aptamers that bind a target (e.g., a protein) involved with the pathology, so that binding of the composition to the target alters the biological function of the target, thereby treating the pathology.

The patient having a pathology, e.g. the patient treated by the methods of this invention can be a mammal, or more particularly, a human.

In practice, the compounds or their pharmaceutically acceptable salts, are administered in amounts which will be sufficient to exert their desired biological activity.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixirs, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Preparation of 3′-5′-di-PEGylated Aptamers

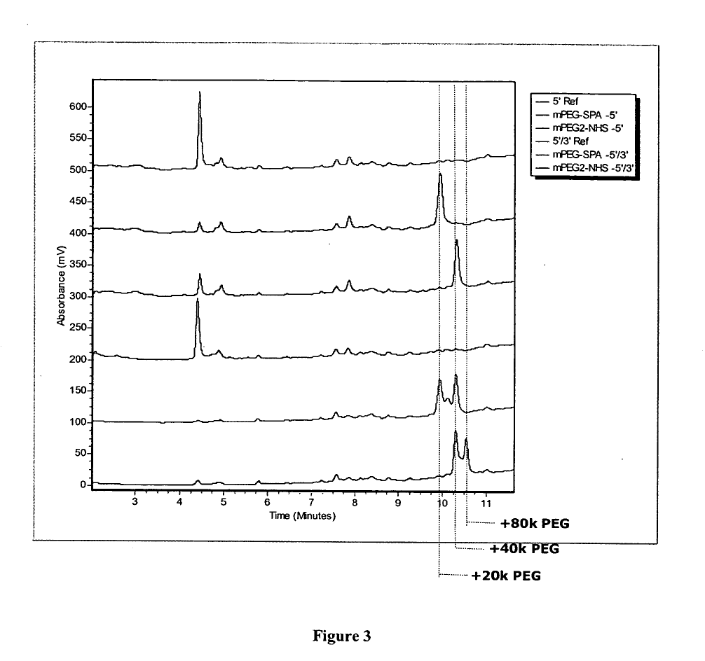

Two oligonucleotides having sequence SEQ ID No.1—5′-NH₂-2′OMe[GGGGUUAUUACAGAGUCUGUAUAGCUGUACCC]-[3′T]-3′—and SEQ ID No. 2—5′-NH₂-2′OMe[GGGGUUAUUACAGAGUCUGUAUAGCUGUACCC]-NH₂-3′—(wherein “2′OMe” indicates modified nucleotides having a methoxy group at the 2′ position and [3′T] refers to an inverted thymidine residue) were synthesized on an Expedite DNA synthesizer (ABI, Foster City, Calif.) according to the recommended manufacturer's procedures using standard commercially available 2′-OMe RNA phosphoramidites (Glen Research, Sterling, Va.). 5′-amine functions were attached with an amino-modifier C6 reagent, and the 3′-amine was introduced using 3′-amino-modifier C3 CPG (Glen Research, Sterling, Va.). After deprotection, the oligonucleotides were evaporated to dryness, ethanol precipitated twice to remove residual ammonia, and re-dissolved in water to a concentration of 1 mM. For the conjugation reactions, 7.5 μL of the oligonucleotide were mixed with an equal volume of 200 mM NaHCO₃-buffer pH 8.3, and 15 μL of mPEG-SPA 20 kDa at a concentration of 40 mg/mL or mPEG-NHS 40 kDa at a concentration of 80 mg/mL in acetonitrile were added. After reacting for 60 minutes at room temperature, 5 μL aliquots of the reactions were quenched with 4 μL 100 mM Tris-buffer, pH 7.4. RP-HPLC analysis was performed on a DNA-Prep HC column (Transgenomic, Omaha, Nebr.), solvent A 100 mM TEAA, solvent B 100 mM TEAA in 90% v/v acetonitrile, 5-100% B in 18 min, column temperature 80° C., injection 10 μL, absorbance detection at 260 nm. The purification HPLC traces are shown in FIG. 3, and indicate the successful preparation of di-PEGylated conjugates with molecular weights up to 80 kDa PEG.

### Example 2

### Bidentate PDGF Aptamers With an Oligonucleotide Splint Stabilizer

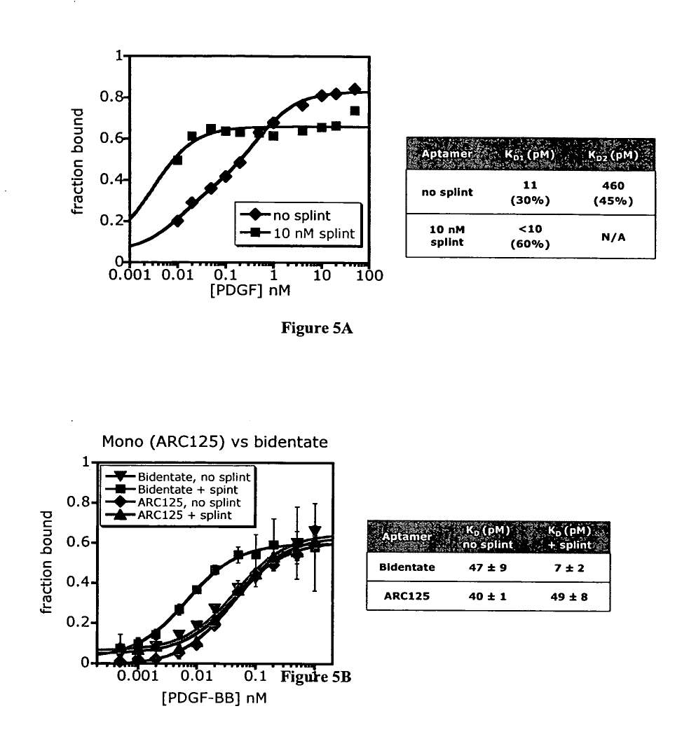

High molecular weight aptamer compositions capable of binding to platelet derived growth factor (PDGF) were produced using the following methods. A dimeric, or bidentate PDGF aptamer having the sequence shown in FIG. 4(A) was synthesized using standard reagents (oligonucleotides supplied by Integrated DNA Technologies, Coralville, Iowa). As shown in FIG. 4(B), the enhanced affinity of the bidentate aptamer to its target (either PDGF BB or AB) was greatest at higher protein concentrations where the binding conditions were 25° C. in 1×PBS with an RNA ligand concentration of <10 pM. In addition, as shown in FIG. 5, the use of a DNA splint complementary to the linker region as illustrated in FIG. 4(A), had an enhancing effect on the affinity of the bidentate aptamer ligand to PDGF-BB as shown in the plots of proportion of bidentate aptamer to PDGF-BB target with and without an oligonucleotide splint. This enhancement could reflect a splint-dependent reduction in rotational degrees of freedom within the linker region, leading to an increase in the effective concentration of the bidentate ligand. The addition of splint DNA to the monovalent aptamer to PDGF-BB had no effect on binding affinity (FIG. 5).

SEQ ID No. 3 - splint oligonucleotide
5′-AAAGGAATTCTACGCCTCGAGTGCAGCCCAGGAACTATTT-3′
SEQ ID No. 4 -- PDGF bidentate aptamer
5′
TACTCAGGGCACTGCAAGCAATTGTGGTCCCAATGGGCTGAGTATGTGGT
CTATG-
TCGTCGTTCGCTAGTAGTTCCTGGGCTGCACTCGAGGCGTAGAATTCCCC
CGATGCGCGCTGTTCTTACTCAGGGCACTGCAAGCAATTGTGGTCCCAAT
GGGCTGAGTAT-3′

### Example 3

### TGFβ2 Chelating Aptamers With Homolymeric Oligonucleotide Linkers

High molecular weight aptamer compositions capable of binding to TGFβ2 were produced using the following methods. Several constructs of TGFβ2 bidentate aptamers based on a TGFβ2 aptamer having the sequence shown below in SEQ ID No. 4 were synthesized with poly U/C linkers of various lengths and sequence compositions as shown in FIG. 6. When linking the aptamers a double helical extension at the 3′ end of the aptamer was added to disrupt irrelevant conformers. Table 1 shows various spacer lengths and sequences that were used in the synthesis of the TGFβ2 bidentate aptamers.

TABLE 1
Linker Sequences, N =
length of oligonucleotide.

| | |
|---|---|
| N | sequence |

| | | |
|---|---|---|
| SEQ ID NO:6 | 5 | UUUUU |
| SEQ ID NO:7 | 10 | UU UCCU UUUU |
| SEQ ID NO:8 | 20 | UU (UCCU)₃CUUUUU |
| SEQ ID NO:9 | 30 | UU (UCCU)₆UUUU |
| SEQ ID NO:10 | 40 | U (UCCU)₈UCUUUUU |
| SEQ ID NO:11 | 50 | U (UCCU)₃UU (UCCU)₇UC (U)₅ |

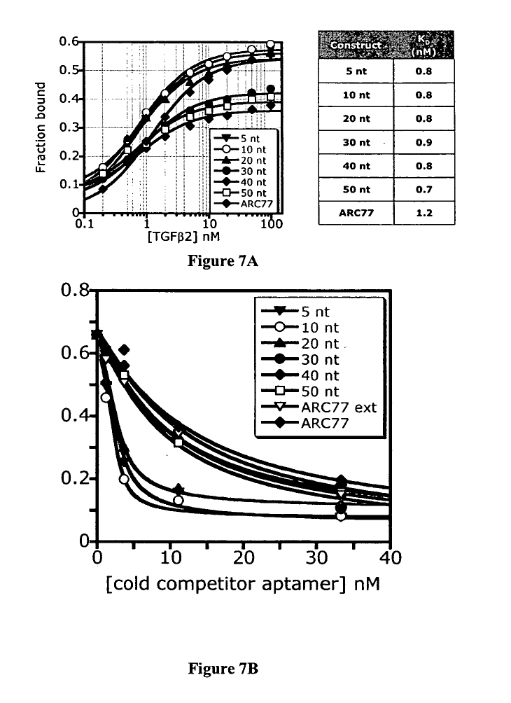

FIG. 7(A) is a binding plot showing the proportion of bidentate aptamer with various linker lengths and compositions and their effect on binding to TGFβ2. The addition of the linker has little effect on binding affinity as reflected in a <2-fold change in K_{D}, but at high TGFβ2 concentrations differences in binding are observed. Under these conditions, binding is linker-length dependent with enhanced binding observed for linkers ≦20 nucleotides.

FIG. 7B is a competitive assay in which all of the bidentate constructs are competing for binding to TGFβ2 with radiolabeled ARC77. Non-labeled ARC77 and non-labeled ARC77 containing a 5-basepair terminal extension (shaded region in FIG. 6) are included as monodentate control competitors. Similar competition is observed for increasing concentrations of control aptamers and bidentate aptamers containing linker regions ≧30 nucleotides. However, bidentate aptamers with shorter linker sequences (≦20 nucleotides) display approximately 10-fold lower K_{comp}, consistent with enhanced binding affinity. Taken together, the results shown in FIGS. 7A and 7B indicate that linkage of aptamers to form a single bidentate aptamer can have desirable effects on ligand binding stability.

SEQ ID No.5 (“f” indicates modified nucleotides having a fluoro group at the 2′ position) 5′-GGAGGfUfUAfUfUAfCAGAGfUfCfUGfUAfUAGfCfUGfUAfCfUfCfC-[3T]-3′

What is claimed is:

1. A high molecular weight aptamer composition comprising:
(a) a nucleic acid comprising two or more aptamers, and
(b) a stabilizing moiety comprising a linking moiety, wherein the linking moiety is not a nucleic acid molecule.

2. The aptamer composition of claim 1, wherein the linking moiety comprises a polyalkylene glycol.

3. The aptamer composition of claim 2, wherein the linking moiety comprises polyethylene glycol (PEG).

4. The aptamer composition of claim 3, wherein the nucleic acid comprises first and second aptamers.

5. The aptamer composition of claim 4, wherein the first and second aptamers are linked by the PEG linking moiety, and further wherein the primary structure of the aptamer composition comprises a linear arrangement in which the first aptamer is linked to a first terminus of the PEG linking moiety and the second aptamer is linked to a second terminus of the PEG linking moiety.

6. The aptamer composition of claim 3, wherein the polyethylene glycol (PEG) linking moiety is multi-activated.

7. The aptamer composition of claim 6, wherein the PEG linking moiety is bi-activated.

8. The aptamer composition of claim 1, wherein the aptamer composition is capable of binding to platelet derived growth factor (PDGF).

9. The aptamer composition of claim 4, wherein the aptamer composition is capable of binding to TGFβ2.

10. The aptamer composition of claim 1, wherein the high molecular weight aptamer composition has a molecular weight selected from the group consisting of greater than 10 kD, greater than 20 kD, greater than 40 kD and greater than 80 kD.

11. A high molecular weight aptamer composition comprising:
(a) a nucleic acid moiety comprising two or more aptamer domains joined by a linker domain, and
(b) a stabilizing moiety comprising one or more polyalkylene glycol moieties attached to the linker domain.

12. The aptamer composition of claim 11, wherein the stabilizing moiety comprises one or more polyethylene glycol (PEG) moieties.

13. The aptamer composition of claim 11, wherein the aptamer composition is capable of binding to platelet derived growth factor (PDGF).

14. The aptamer composition of claim 11, wherein the aptamer composition is capable of binding to TGFβ2.

15. The aptamer composition of claim 11, wherein the high molecular weight aptamer composition has a molecular weight selected from the group consisting of greater than 10 kD, greater than 20 kD, greater than 40 kD and greater than 80 kD.

16. A high molecular weight aptamer composition comprising:
(a) a nucleic acid comprising two or more aptamer domains and a linker domain, and
(b) a stabilizing moiety comprising an oligonucleotide splint which hybridizes to at least a portion of the linker domain, wherein the oligonucleotide splint comprises a nucleotide sequence having at least 40 nucleotides.

17. The aptamer composition of claim 16, wherein the oligonucleotide splint hybridizes to at least 20 nucleotides of the linker domain.

18. The aptamer composition of claim 16, wherein the aptamer composition is capable of binding to platelet derived growth factor (PDGF).

19. The aptamer composition of claim 16, wherein the aptamer composition is capable of binding to TGFβ2.

20. The aptamer composition of claim 16, wherein the high molecular weight aptamer composition has a molecular weight selected from the group consisting of greater than 10 kD, greater than 20 kD, greater than 40 kD and greater than 80 kD.

21. A high molecular weight aptamer composition comprising:
(a) a nucleic acid moiety comprising two or more aptamer domains and a linker domain, and
(b) a stabilizing moiety comprising an oligonucleotide splint that hybridizes to at least a portion of the linker domain, wherein the oligonucleotide splint has one or more polyalkylene glycol moieties attached thereto.

22. The aptamer composition of claim 16, wherein the oligonucleotide splint hybridizes to at least 20 nucleotides of the linker domain.

23. The aptamer composition of claim 21, wherein the oligonucleotide splint has one or more polyethylene glycol (PEG) moieties attached thereto.

24. The aptamer composition of claim 21, wherein the oligonucleotide splint comprises a nucleotide sequence having at least 40 nucleotides.

25. The aptamer composition of claim 21, wherein the aptamer composition is capable of binding to platelet derived growth factor (PDGF).

26. The aptamer composition of claim 21, wherein the aptamer composition is capable of binding to TGFβ2.

27. The aptamer composition of claim 21, wherein the high molecular weight aptamer composition has a molecular weight selected from the group consisting of greater than 10 kD, greater than 20 kD, greater than 40 kD and greater than 80 kD.

28. A high molecular weight aptamer composition comprising:
(a) a nucleic acid moiety comprising two or more aptamer domains and a linker domain, and
(b) a stabilizing moiety comprising an oligonucleotide splint which hybridizes to at least a portion of the linker domain,
wherein at least one of the two or more aptamer domains is in the unbound state.

29. The aptamer composition of claim 28, wherein the oligonucleotide splint hybridizes to at least 20 nucleotides of the linker domain.

30. The aptamer composition of claim 28, wherein the oligonucleotide splint comprises a nucleotide sequence having at least 40 nucleotides.

31. The aptamer composition of claim 30, wherein the oligonucleotide splint has one or more polyalkylene glycol moieties attached thereto.

32. The aptamer composition of claim 31, wherein the oligonucleotide splint has one or more polyethylene glycol (PEG) moieties attached thereto.

33. The aptamer composition of claim 28, wherein the aptamer composition is capable of binding to platelet derived growth factor (PDGF).

34. The aptamer composition of claim 28; wherein the aptamer composition is capable of binding to TGFβ2.

35. The aptamer composition of claim 28, wherein the high molecular weight aptamer composition has a molecular weight selected from the group consisting of greater than 10 kD, greater than 20 kD, greater than 40 kD and greater than 80 kD.

36. A therapeutic composition comprising the aptamer composition of claim 1.

37. A therapeutic composition comprising the aptamer composition of claim 11.

38. A therapeutic composition comprising the aptamer composition of claim 16.

39. A therapeutic composition comprising the aptamer composition of claim 21.

40. A therapeutic composition comprising the aptamer composition of claim 28.

41. A high molecular weight aptamer composition comprising:
(a) an aptamer, and
(b) two or more non-nucleic acid stabilizing moieties.

42. The aptamer composition of claim 41, wherein the stabilizing moieties comprise a polyalkylene glycol.

43 The aptamer composition of claim 42, wherein the stabilizing moieties comprise polyethylene glycol (PEG).

44. The aptamer composition of claim 41, wherein the aptamer is multi-activated.

45. The aptamer composition of claim 44, wherein the aptamer is bi-activated.

46. A method of improving the pharmacokinetic or pharmacodynamic properties of an aptamer therapeutic composition comprising the steps of introducing reactive groups in a nucleic acid aptamer, reacting the reactive groups on the aptamer with reactive groups on a stabilizing moiety, thereby forming a stabilized high molecular weight therapeutic aptamer.

47. The method of claim 46 wherein the reactive groups on the aptamer composition are amino groups at 5′ or 3′ ends of the aptamer introduced by modified phosphoramidite synthesis.

48. The method of claim 46 wherein the stabilizing moiety is polyethylene glycol (PEG).

49. The method of claim 48 wherein the PEG is homo-bifunctional and the resulting aptamer is a dimer linked by a PEG linker.

50. The method of claim 46 wherein the aptamer is multiply activated.

51. The method of claim 50 wherein the aptamer is bi-activated at 5′ and 3′ termini.

52. The method of claim 50 wherein the stabilizing moiety is a mono-activated PEG and the resulting aptamer is bi-PEGylated.

53. A method of treating disease in a subject comprising the steps of administering a therapeutically effective amount of a high molecular weight aptamer composition of claim 1.

54. A method of treating disease in a subject comprising the steps of administering a therapeutically effective amount of a high molecular weight aptamer composition of claim 11.

55. A method of treating disease in a subject comprising the steps of administering a therapeutically effective amount of a high molecular weight aptamer composition of claim 16.

56. A method of treating disease in a subject comprising the steps of administering a therapeutically effective amount of a high molecular weight aptamer composition of claim 21.

57. A method of treating disease in a subject comprising the steps of administering a therapeutically effective amount of a high molecular weight aptamer composition of claim 28.


SEQUENCE LISTING (13 sequences)

SEQ ID NO: 1 - RNA, 32 nt, Artificial
  synthetic aptamer
gggguuauuacagagucuguauagcuguaccc

SEQ ID NO: 2 - RNA, 32 nt, Artificial; identical to SEQ ID NO: 1
  synthetic aptamer

SEQ ID NO: 3 - DNA, 40 bp, Artificial
  synthetic aptamer
aaaggaattctacgcctcgagtgcagcccaggaactattt

SEQ ID NO: 4 - DNA, 166 bp, Artificial
  synthetic aptamer
tactcagggcactgcaagcaattgtggtcccaatgggctgagtatgtggtctatgtcgtcgttcgctagtagttcctgggctgcactcgaggcgtagaattcccccgatgcgcgctgttcttactcagggcactgcaagcaattgtggtcccaatgggctgagtat

SEQ ID NO: 5 - RNA, 34 nt, Artificial
  synthetic aptamer
ggaggnnannanagagnnngnanagnngnannnn

SEQ ID NO: 6 - RNA, 5 nt, Artificial
  synthetic aptamer
uuuuu

SEQ ID NO: 7 - RNA, 9 nt, Artificial
  synthetic aptamer
uuuccuuuu

SEQ ID NO: 8 - RNA, 20 nt, Artificial
  synthetic aptamer
uuuccuuccuuccucuuuuu

SEQ ID NO: 9 - RNA, 30 nt, Artificial
  synthetic aptamer
uuuccuuccuuccuuccuuccuuccuuuuu

SEQ ID NO: 10 - RNA, 40 nt, Artificial
  synthetic aptamer
uuccuuccuuccuuccuuccuuccuuccuuccuucuuuuu

SEQ ID NO: 11 - RNA, 50 nt, Artificial
  synthetic aptamer
uuccuuccuuccuuuuccuuccuuccuuccuuccuuccuuccuucuuuuu

SEQ ID NO: 12 - RNA, 34 nt, Artificial
  synthetic aptamer
ggagguuauuacagagucuguauagcuguacucc

SEQ ID NO: 13 - RNA, 42 nt, Artificial
  synthetic aptamer
gcgccgagguuauuacagagucuguauagcuguacucggcgc